(19) United States
(12) Patent Application Publication
(10) Pub. No.: US 2026/0074029 A1
(43) Pub. Date: Mar. 12, 2026

(54) SYSTEM AND METHOD FOR CLINICAL TRIALS

(71) Applicant: Muehlhausen Limited, Cloughjordan (IE)

(72) Inventor: Wilhelm MUEHLHAUSEN, Cloughjordan (IE)

(21) Appl. No.: 19/298,345

(22) Filed: Aug. 13, 2025

Related U.S. Application Data
(63) Continuation of application No. 17/531,839, filed on Nov. 22, 2021, now abandoned.
(60) Provisional application No. 63/117,472, filed on Nov. 24, 2020.

(51) Int. Cl. G16H 10/20; G16H 40/67; G16H 50/20; H04L 9/40

(52) U.S. Cl. CPC G16H 10/20; G16H 40/67; G16H 50/20; H04L 63/10

(57) ABSTRACT

A system and method for automatically constructing patient-facing protocols from an initial clinical trial protocol design. The patient-facing protocols preferably include one or more wearables or other sensors (including sensors in the phone), for monitoring patient behaviors. The patient-facing protocols may also include one or more questions to be asked of the patient, for subject patient state information. Such a system is preferably able to both construct an effective patient-facing protocol for a clinical trial, including any aspects that may have at least some flexibility, such as for example a period of time during which a particular action may be taken. Such flexibility is preferably then automatically incorporated as the system monitors the behavior of each patient, for example by adjusting a time that an alert or alarm is sent to a particular patient within the permitted period on a daily, weekly, monthly or yearly basis, or according to any other permitted period of time.

### FIELD OF THE INVENTION

The present invention relates to a system and method for clinical trials and remote patient monitoring, and in particular, to such a system and method for automatically constructing patient-facing protocols and monitoring same.

### BACKGROUND OF THE INVENTION

US Patent Application No. US20200202984 relates to a system and method for building intuitive clinical trial applications. The '984 application relates to creating customized applications for patients to interact with during clinical trials. However, this application only suggests creation of very basic software for patients to interact with, for example by answering questions. The problem with such art known systems is that they are unable to rapidly construct clinical trial protocols and to analyze such protocols for potential problems. Currently, art known systems rely on human effort which is time consuming and expensive, and is prone to error.

### BRIEF SUMMARY OF THE INVENTION

The background art does not teach or suggest a system or method for automatically constructing patient-facing protocols from an initial clinical trial protocol design. The background art also does not teach or suggest a system or method for monitoring the behaviors of patients during a clinical trial.

The present invention overcomes the drawbacks of the background art by providing, in at least some embodiments, a system and method for automatically constructing patient-facing protocols from an initial clinical trial protocol design. The patient-facing protocols preferably include one or more wearables or other sensors (including sensors in the phone), for monitoring patient behaviors. The patient-facing protocols may also include one or more questions to be asked of the patient, for subject patient state information. Such a system is preferably able to both construct an effective patient-facing protocol for a clinical trial, including any aspects that may have at least some flexibility, such as for example a period of time during which a particular action may be taken. Such flexibility is preferably then automatically incorporated as the system monitors the behavior of each patient, for example by adjusting a time that an alert or alarm is sent to a particular patient within the permitted period on a daily, weekly, monthly or yearly basis, or according to any other permitted period of time.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Unless otherwise defined, all technical and scientific terms used herein have the same meaning as commonly understood by one of ordinary skill in the art to which this invention belongs. The materials, methods, and examples provided herein are illustrative only and not intended to be limiting.

An algorithm as described herein may refer to any series of functions, steps, one or more methods or one or more processes, for example for performing data analysis.

Implementation of the apparatuses, devices, methods and systems of the present disclosure involve performing or completing certain selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be implemented as a number of software instructions being executed by a computer (e.g., a processor of the computer) using an operating system. In any case, selected steps of methods of at least some embodiments of the disclosure can be described as being performed by a processor, such as a computing platform for executing a plurality of instructions. The processor is configured to execute a predefined set of operations in response to receiving a corresponding instruction selected from a predefined native instruction set of codes.

Software (e.g., an application, computer instructions) which is configured to perform (or cause to be performed) certain functionality may also be referred to as a “module” for performing that functionality, and also may be referred to a “processor” for performing such functionality. Thus, processor, according to some embodiments, may be a hardware component, or, according to some embodiments, a software component.

Further to this end, in some embodiments: a processor may also be referred to as a module; in some embodiments, a processor may comprise one or more modules; in some embodiments, a module may comprise computer instructions—which can be a set of instructions, an application, software—which are operable on a computational device (e.g., a processor) to cause the computational device to conduct and/or achieve one or more specific functionality. Some embodiments are described with regard to a “computer,” a “computer network,” and/or a “computer operational on a computer network.” It is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a tablet or phablet, including without limitation an iPad, a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A-1C relate to exemplary, illustrative, non-limiting systems according to at least some embodiments of the present invention;

FIG. 2 relates to an exemplary, illustrative, non-limiting AI analysis engine, for example for implementation with the system of any of FIGS. 1A-1C, according to at least some embodiments of the present invention;

FIGS. 3A-3C relate to exemplary, illustrative, non-limiting AI engines according to at least some embodiments of the present invention;

FIG. 4 relates to a further exemplary, illustrative, non-limiting system according to at least some embodiments;

FIG. 5 relates to a non-limiting, exemplary training method for an AI engine as described herein;

FIGS. 6 and 7 relate to exemplary, illustrative, non-limiting systems incorporating blockchain according to at least some embodiments of the present invention; and

FIG. 8 shows an exemplary, non-limiting flow for a clinical trial protocol design studio.

### DESCRIPTION OF AT LEAST SOME EMBODIMENTS

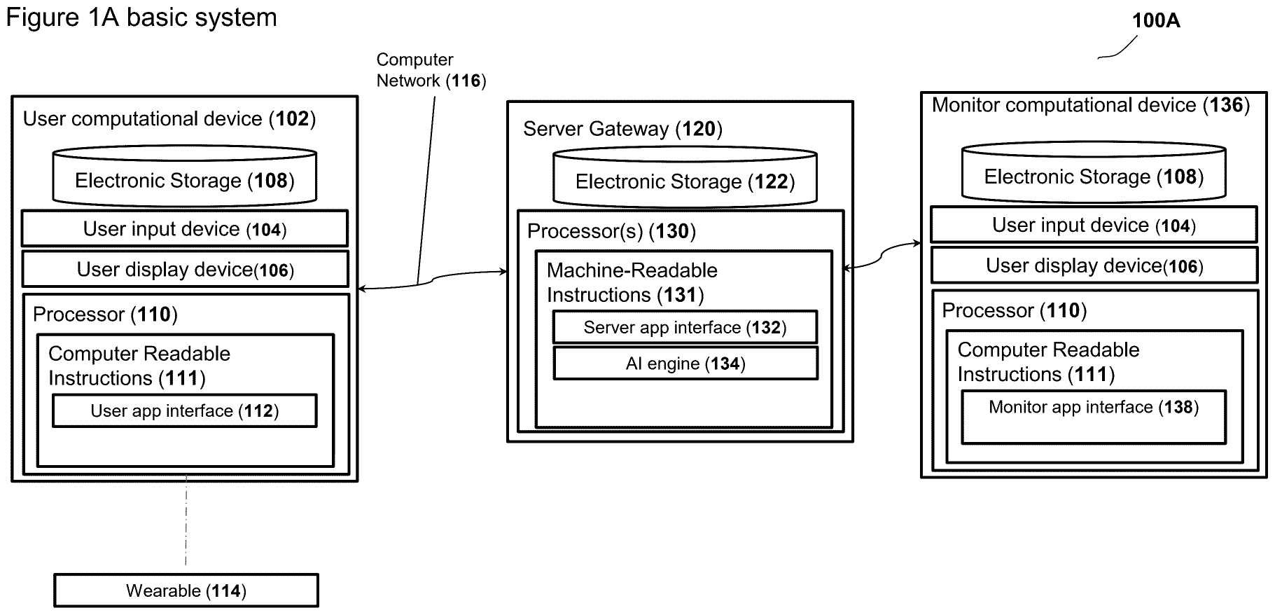

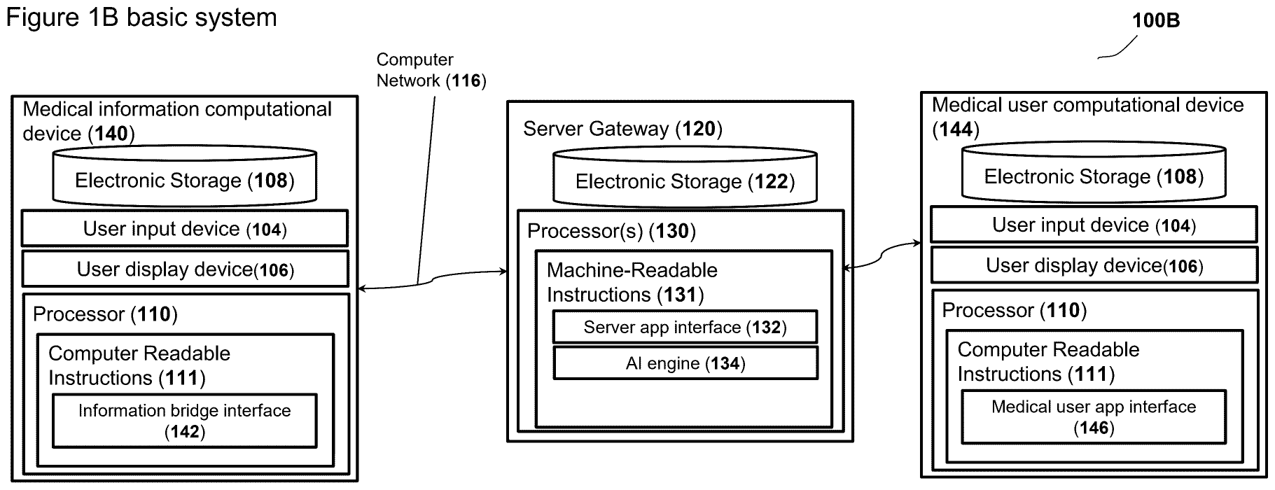

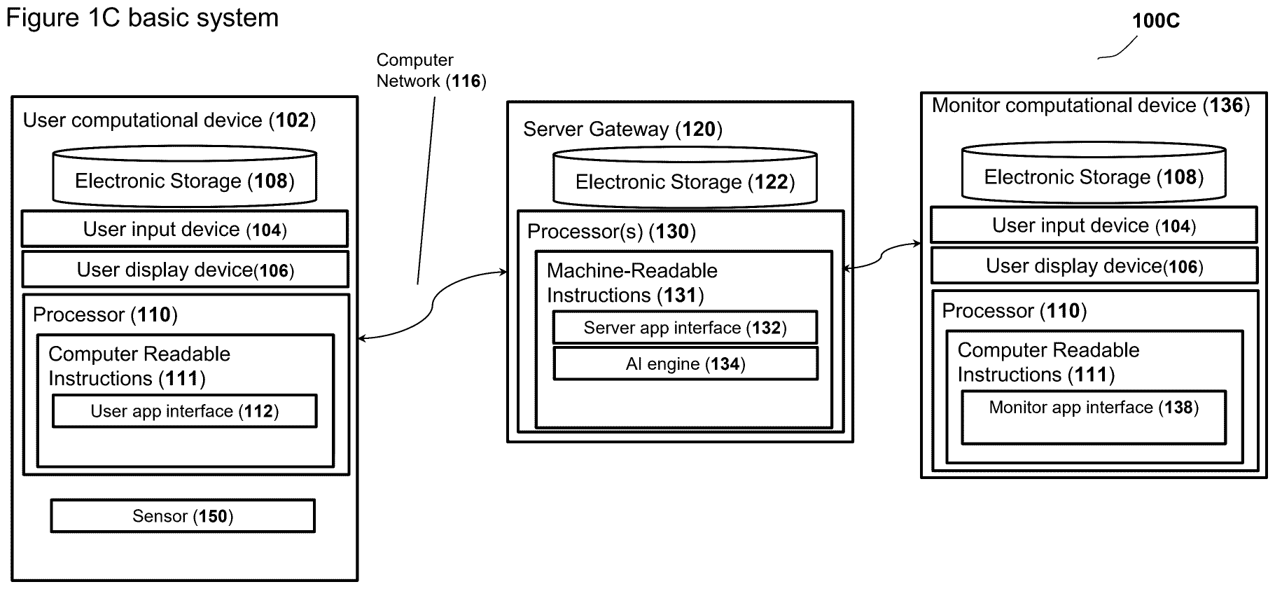

FIGS. 1A-1C relate to exemplary, illustrative, non-limiting systems according to at least some embodiments of the present invention. FIG. 1A shows an exemplary system 100A, featuring a user computational device 102 for being operated by a patient. In this non-limiting example, the patient would wear or otherwise be in physical communication with a wearable 114, which may be in wired or wireless communication with user computational device 102, whether continuously or periodically. Wearable 114 comprises one or more sensors (not shown) which may monitor one or more behaviors or physiological parameters (i.e. blood pressure) of the patient. Wearable 114 may also issue one or more alarms, to remind the patient to engage in certain behaviors or alternatively to desist from engaging in one or more behaviors. Additionally, or alternatively, user computational device 102 may issue such alarms. Wearable 114 may also receive manual inputs from the patient. Additionally or alternatively, user computational device 102 may receive such manual inputs.

Information from wearable 114 is preferably transmitted to user computational device 102 and then to a server gateway 122. Alternatively, such information may be transmitted directly to server gateway 122. User computational device 102 may also transmit additional information to, and receive information from, server gateway 122. Such communication may occur for example through a computational network 116, which may comprise the internet for example.

The information transmitted from wearable 114 and/or user computational device 102 may for example comprise sensor data regarding one or more behaviors or physiological parameters of the patient. The information may also comprise one or more subjective answers to questions from the patient, to provide subjective information about the state of the patient. This information is preferably transmitted to server gateway 122 to monitor the patient during the clinical trial. The received information may be analyzed for example by an AI engine 134 and may be received through a server app interface 132. AI engine 134 may for example analyze the information to determine whether the patient is complying with one more aspect of the protocol, and/or to detect any anomalies which may relate to side effects or other undesirable effects of following the protocol. AI engine 134 may also determine that information is to be transmitted to user computational device 102 and/or wearable 114, for example in the form of questions, prompts, alarms, adjustments to the protocol and so forth.

Information that is transmitted to user computational device 102 may be displayed through a user app interface 112. Optionally, answers to questions and other input from the patient may be received through user app interface 112. Alarms and prompts may be invoked through user app interface 112. Adjustments to the patient interaction, for example through wearable 114, are preferably also controlled through user app interface 112. It should be noted that although reference is made throughout to such actions being taken by the patient, it may be that such interactions are performed by a caregiver instead.

User computational device 102 also comprises a processor 110 and a memory 111. Functions of processor 110 preferably relate to those performed by any suitable computational processor, which generally refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory, such as a memory 111 in this non-limiting example. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Also optionally, memory 111 is configured for storing a defined native instruction set of codes. Processor 110 is configured to perform a defined set of basic operations in response to receiving a corresponding basic instruction selected from the defined native instruction set of codes stored in memory 111. For example and without limitation, memory 111 may store a first set of machine codes selected from the native instruction set for receiving information from the user through user app interface 104, such as a response to a prompt or an answer to a subjective question for example, and a second set of machine codes selected from the native instruction set for transmitting such information to server gateway 120 as patient response information. Memory 111 may store a third set of machine codes selected from the native instruction set for receiving data from wearable 114, and a fourth set of machine codes selected from the native instruction set for transmitting such data to server gateway 120 as wearable data.

Similarly, server gateway 120 preferably comprises processor 130 and memory with machine readable instructions 131 with related or at least similar functions, including without limitation functions of server gateway 120 as described herein. For example and without limitation, memory 131 may store a first set of machine codes selected from the native instruction set for receiving sensor data and/or patient subjective information from user computational device 102, and a second set of machine codes selected from the native instruction set for executing functions of AI engine 134. Memory 131 may store a third set of machine codes selected from the native instruction set for transmitting one or more prompts or alarms to user computational device 102.

Non-limiting examples of wearable 114 include an accelerometer, a step counter, a GPS or equivalent sensor, actigraphy devices, insoles with one or more sensors, pulse oximeter, heartrate monitor, an airflow peak flow measuring device, brain wave monitor (EEG), an ECG (electrocardiogram). Optionally wearable 114 may comprise one or more devices which are not actually worn but which are suitable for obtaining a physiological measurement, such as for example and without limitation electronic weighing scales, blood glucose or other blood component measurement device, a needle tracking and/or disposal monitoring device, an ingestible sensor, and/or environmental monitors including but not limited to movement triggered cameras or sensors, air quality measurement devices, refrigerators that monitor food withdrawal or insertion, or video monitors.

System 100A also preferably features a monitor computational device 136, for monitoring the behaviors and/or subjective answers of the patient as received through wearable 114 and/or user computational device 102. Monitor computational device 136 may for example be operated by medical personnel who are monitoring the patient(s) enrolled in the clinical trial or remote monitoring healthcare. Such personnel may interact with the information received by server gateway 120, including without limitation analysis by AI engine 134, through a monitor app interface 138. For example and without limitation, through monitor app interface 138, the medical personnel could monitor compliance with the clinical trial protocol and/or health state change, including without limitation such symptoms as fever, lethargy, weakness, and/or specific diseases such as COPD, asthma, transplant rejection and the like. AI engine 134 may monitor these issues or potential problems, and then inform the medical personnel through monitor app interface 138. Optionally monitor app interface 138 is used for manual monitoring by medical personnel.

FIG. 1B shows an exemplary system 100B, now featuring a medical information computational device 140 for providing medical information for constructing a clinical trial protocol. Such medical information may relate to requirements of a protocol, endpoints to be determined, data to be measured and so forth. The minimal requirements of protocol structure are described in the ICH Guideline for Good Clinical Practice ICH E6 (R2) (https://ichgcp.net/6-clinical-trial-protocol-and-protocol-amendments). The information may be obtained from one or more medical records, databases regarding clinical trial practice and so forth, preferably through an information bridge interface 142. Some data may be obtained as scanned paper documents, such that OCR (optical character recognition) may be required for converting typed, handwritten or printed text images into machine-encoded text. Non-limiting examples of suitable processes include preprocessing the image to correct such issues as skew; layout analysis to detect words and other information; and optionally an analysis to turn such information to text or other appropriate information.

The information is preferably then transmitted through server gateway 120 to a medical user computational device 144, for being operated by medical personnel for constructing the clinical trial protocol. Medical user computational device 144 preferably comprises a medical user app interface 146, for interacting with the medical personnel who are constructing the clinical trial protocol, for example according to the above reference.

FIG. 1C shows an exemplary system 100C, now featuring an alternative embodiment of user computational device 102, comprising a sensor 150. For example, user computational device 102 may comprise a cellular telephone or mobile communication device, which may feature one or more sensors in an off the shelf implementation. A non-limiting example of such a sensor would be an IMU, a gyroscope, an accelerometer, a GPS and so forth. Optionally a combination of such sensors would be present. Such one or more sensors may be used to monitor one or more behaviors of the patient in an automated and unobtrusive manner. Preferably, the patient would have user computational device 102 in their personal possession, for example by being carried by the patient. The implementations of FIGS. 1A and 1C may be combined.

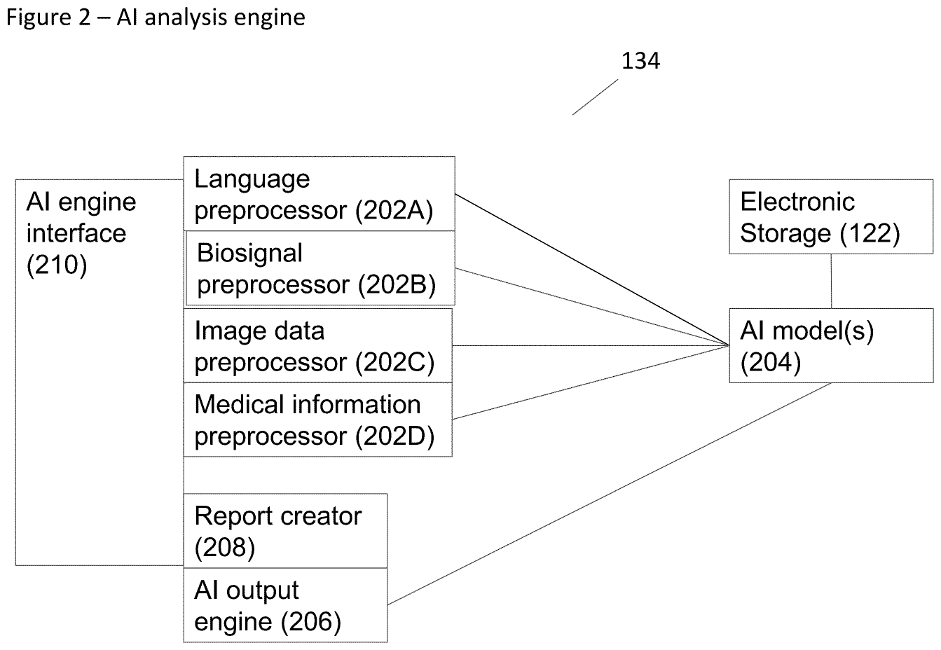

FIG. 2 relates to an exemplary, illustrative, non-limiting AI analysis engine, for example for implementation with the system of any of FIGS. 1A-1C, according to at least some embodiments of the present invention. As shown in a detailed implementation of AI analysis engine 134, preferably a plurality of preprocessing components 202 are present as shown, for receiving input data and preprocessing it for further analysis. Non-limiting examples of such preprocessing components 202 include a language preprocessor 202A for receiving natural language inputs, whether from spoken or textual language, from the patient; a biosignal preprocessor 202B, for receiving biosignal data inputs, for example from the previously described sensor(s) and/or wearable; an image data preprocessor 202C; and a medical information preprocessor 202D, for receiving medical information from the patient. Image data preprocessor 202C may be used for medical images taken from medical devices, such as an X-ray, PET scan, CAT scan and the like; and/or from images taken from an optical camera, including without limitation a mobile communication device camera, which may for example by taken by the patient, a caregiver and/or medical personnel. For example, such an optical camera image may be used for monitoring a skin condition of a patient. Medical information preprocessor 202D may receive information regarding answers to a questionnaire, that the patient fills in and submits. Optionally such information relates to metadata around the act of filling in the questionnaire (time, duration, number of breaks during questionnaire interaction, location, situational context awareness via reading of phone sensors, such as for example whether the patient is on call or reading email). The medical record may be used to provide information about how patients get on with their life and their co-morbidities. i.e. a diabetes patient often suffers from other diseases too, i.e. high blood pressure, obesity.

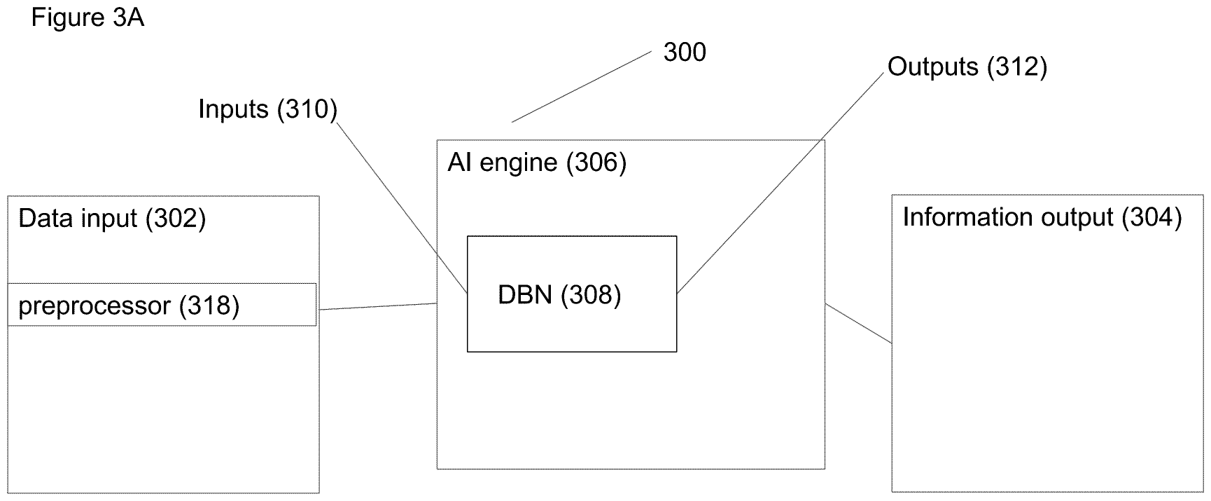

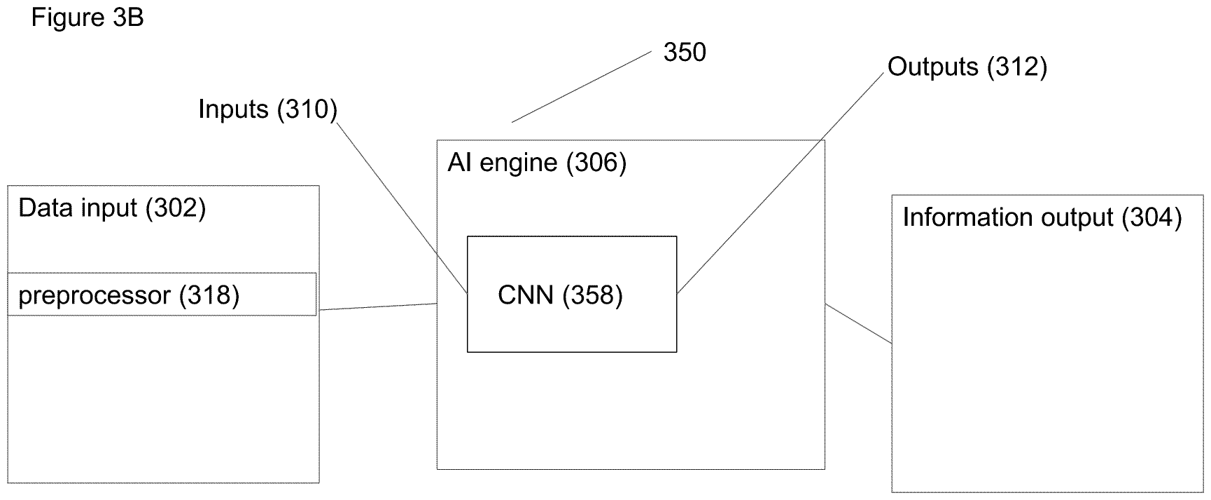

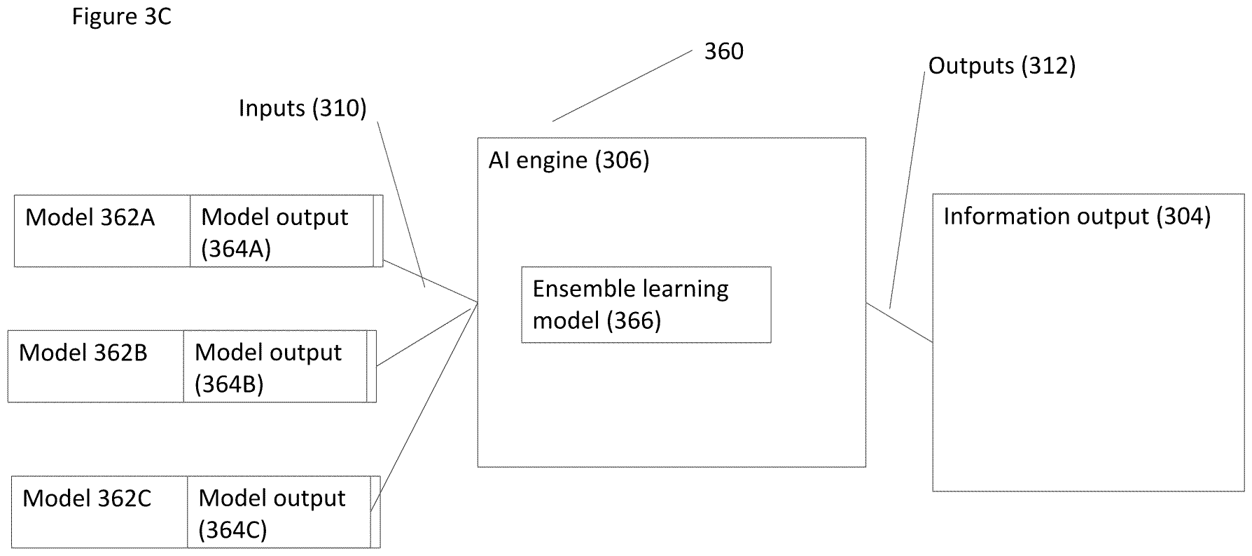

After preprocessing, data is then fed to one or more AI models 204. Optionally each AI model receives a different preprocessed data type; alternatively or additionally, data of more than one type is fed to the same AI model. Non-limiting examples of AI models are described with regard to FIG. 3.

AI model 204 may retrieve additional information from, or store analysis results at electronic storage 122. AI model 204 also preferably analyzes the information with regard to any permitted flexibility in the patient-facing aspects of the clinical trial protocol. The patient-facing protocol for a clinical trial determines any aspects that may have at least some flexibility, such as for example a period of time during which a particular action may be taken. Such flexibility is preferably then automatically incorporated as AI model 204 analyzes incoming data, including with regard to the behavior of each patient. AI model 204 may then make one or more adjustments in real time, for example by adjusting a time that an alert or alarm is sent to a particular patient within the permitted period on a daily, weekly, monthly or yearly basis, or according to any other permitted period of time. Optionally such adjustments may require permission from medical personnel, in at least some cases. After analysis, the results may be transmitted to one or more external computational devices (not shown), for example to invoke an alarm or open a questionnaire or request some action by the patient such as call a medical professional, take a medication, take a sample (i.e. blood, urine, stool, saliva), take a picture, conduct an exercise (i.e. move, run, walk), take a psychometric test (i.e. gambling test).

An AI output engine 206 preferably then receives the analysis from AI model 204 and prepares one or more output actions as previously described. Optionally, a report may be separately prepared by a report creator 208, for example to give information or insights regarding the progress of the clinical trial. Output actions and optionally the report may be output through an AI engine interface 210, through which the previously described input data may also be received.

FIGS. 3A-3C relate to exemplary, illustrative, non-limiting AI engines according to at least some embodiments of the present invention.

Before being fed to such engines, the information has preferably previously been preprocessed according to a method that is known in the art. For example and without limitation, for human language, such preprocessing may occur by tokenization, followed by analysis by a machine learning or deep learning algorithm. A tokenizer is able to break down the text inputs into parts of speech. It is preferably also able to stem the words. For example, running and runs could both be stemmed to the word run. Optionally, the tokenizer operates only to separate words, with or without parts of speech. Each “word” may be defined in a plurality of ways, including but not limited to according to spaces, punctuation (including but not limited to commas, semicolons, colons, periods, apostrophes, quotation marks and the like), symbols (including but not limited to dashes, parentheses and the like), a number of characters in a moving window or a separated window (optionally combined with any of the previous definitions). By “separated window” it is meant that for example n characters are taken into the window, and then the window moves from 2 to n characters to take the next window. A moving window preferably means that each window is separated by 1 character. Optionally character encoding is used, for example for the CNN described embodiment.

Various types of NLP (natural language processing) algorithms may be used in place of, or in combination with, any of the AI models as described herein. Non-limiting examples of such algorithms may include In a system 1080, AI engine 1006 now features combined embeddings 1082, which preferably include a number of different types of trained embedding algorithms. These algorithms may include word2vec, Fasttext and/or sentence2vec. These algorithms are preferably trained on a suitable document corpus. Optionally heuristics may be employed, for example to better the performance of any of the embedding algorithms. As a non-limiting example, word2vec 1082 may be trained on a suitable document corpus as described herein, preferably after some text preprocessing (such as lowercasing the words, removing stop words, commas and other non-essential punctuation/symbols). Other non-limiting examples include the BERT family of deep learning models, including but not limited to BERT, ROBERTa, SentenceBERT and the like.

Turning now to FIG. 3A as shown in a system 300, data inputs 302 are provided and are preprocessed, for example by being tokenized with a tokenizer, at 318. The preprocessed data is then fed into an AI engine 306 and analyzed information 304 is then output. In this non-limiting example, AI engine 306 comprises a DBN (deep belief network) 308. DBN 308 features input neurons 310, a neural network and then outputs 312.

A DBN is a type of neural network composed of multiple layers of latent variables (“hidden units”), with connections between the layers but not between units within each layer.

FIG. 3B relates to a non-limiting exemplary system 350 with similar or the same components as FIG. 3A, except for the neural network model. In this case, AI engine 306 includes a model that is embodied in a CNN (convolutional neural network) 358, which is a different model than that shown in FIG. 3A.

A CNN is a type of neural network that features additional separate convolutional layers for feature extraction, in addition to the neural network layers for classification/identification. Overall, the layers are organized in 3 dimensions: width, height and depth. Further, the neurons in one layer do not connect to all the neurons in the next layer but only to a small region of it. Lastly, the final output will be reduced to a single vector of probability scores, organized along the depth dimension. It is often used for audio and image data analysis, but has recently been also used for natural language processing (NLP; see for example Yin et al, Comparative Study of CNN and RNN for Natural Language Processing, arXiv:1702.01923v1 [cs.CL] 7 Feb. 2017).

FIG. 3C shows a non-limiting exemplary system 360 with an ensemble learning model 366. As inputs, ensemble learning model 366 receives the outputs of a plurality of AI models 362, shown as AI models 362A-362C. Such models may comprise any suitable AI model, for example an AI model as described herein. AI models 362A-362C output model outputs 364A-364C, respectively; model outputs 364 in term form the inputs to ensemble learning model 366, which then in turn provides information output 304. Various types of suitable ensemble learning models 366 are known in the art and may be implemented herein.

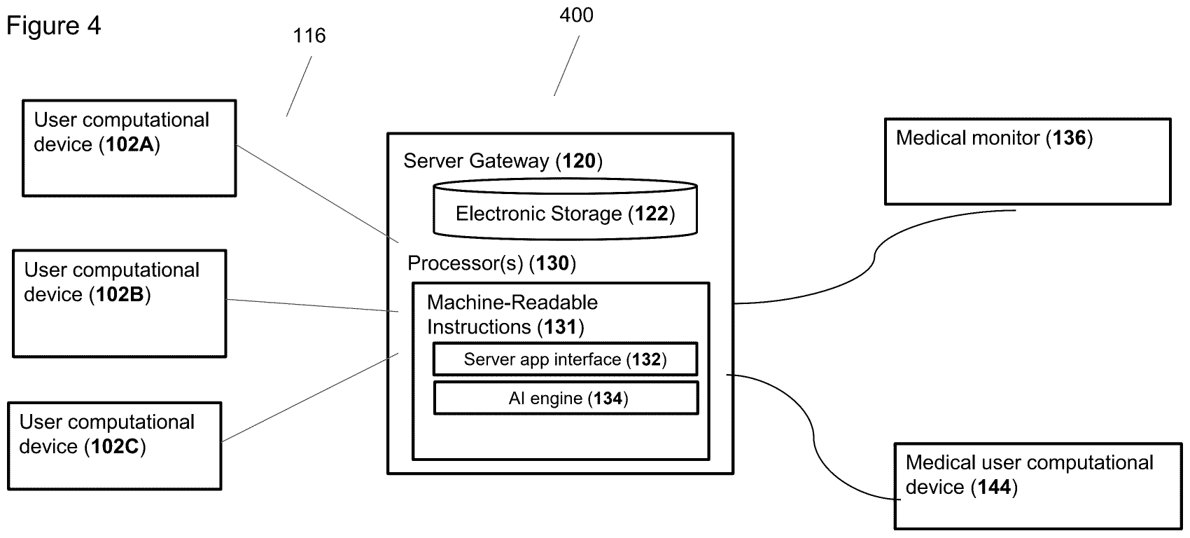

FIG. 4 relates to a further exemplary, illustrative, non-limiting system according to at least some embodiments. The components are the same or similar to those of FIGS. 1A-1C, but in this non-limiting example, a plurality of patients is being monitored through a plurality of user computational devices 102A-102C. Each such user computational device 102A-102C preferably is in communication with a wearable and/or comprises one or more sensors (not shown).

Medical monitor 136 and also medical user computational device 144 are preferably able to access sensor and/or wearable data, and/or are preferably able to access subjective response information from patients as received from and/or through user computational devices 102A-102C. Preferably, monitoring is also provided through AI engine 134 of server gateway 120, for example to automatically alert medical personnel through medical monitor 136 and/or medical user computational device 144 to an issue with a particular patient. Optionally AI engine 134 also notes a change in condition of the patient, such as a worsening of one or more symptoms, or of a disease or physiological condition generally, and sends such an alert. Optionally AI engine 134 may send an alert to the patient, or to both the patient and medical personnel. AI engine 134 may also send a recommended course of action to the patient, a caregiver and/or medical personnel. AI engine 134 may also recommend one or more changes within certain parameters where flexibility is permitted as described herein, for example with regard to a period of time as when a patient may engage in a certain behavior. AI engine 134 may then send a reminder or an alert at a particular point in that period of time which has been shown to be most likely to invoke the desired behavior in that particular patient.

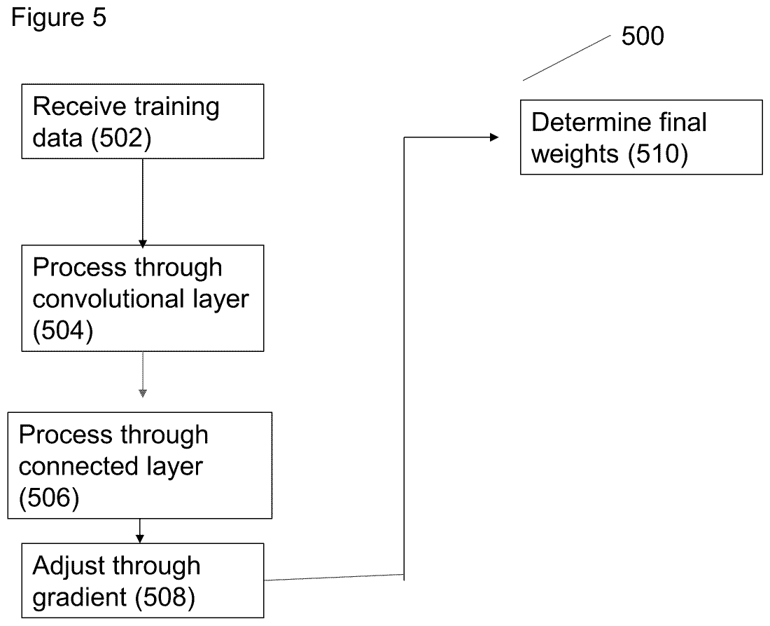

FIG. 5 relates to a non-limiting, exemplary training method for an AI engine as described herein. As shown with regard through flow 500, the training data is received in 502 and it is processed through the convolutional layer of the network in 504. Such processing occurs if a convolutional neural net is used, which is the assumption for this non-limiting example. After that the data is processed through the connected layer in 506 and adjust according to a gradient in 508. Typically, a steep descent gradient is used in which the error is minimized by looking for a gradient. One advantage of this is it helps to avoid local minima where the AI engine may be trained to a certain point but may be in a minimum which is local but it's not the true minimum for that particular engine. The final weights are then determined in 510 after which the model is ready to use.

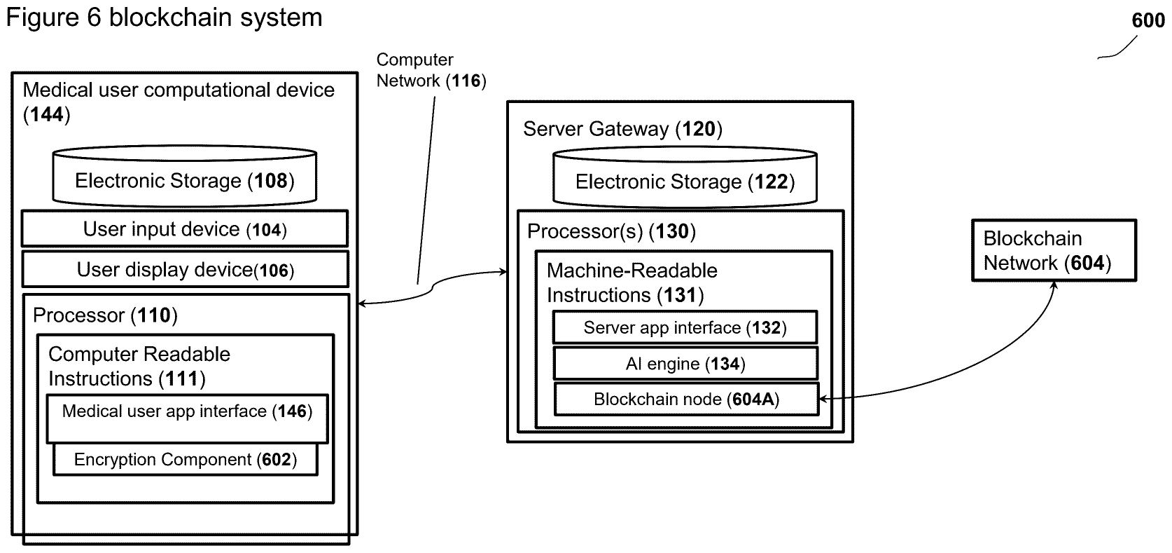

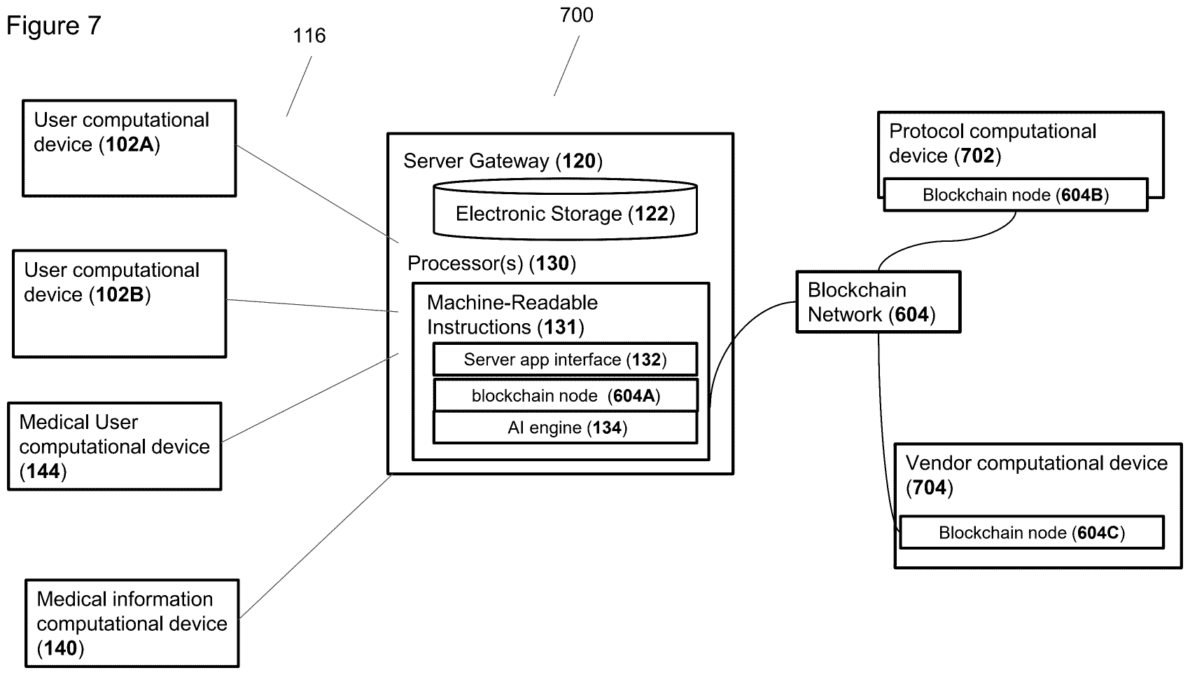

FIGS. 6 and 7 relate to exemplary, illustrative, non-limiting systems incorporating blockchain according to at least some embodiments of the present invention.

FIG. 6 shows an exemplary, illustrative, non-limiting system incorporating blockchain according to at least some embodiments of the present invention. In a system 600, medical user computational device 144 again communicates with server gateway 120 through computational network 116, as shown with regard to FIG. 1B. Components having the same reference number have the same or at least similar function as for FIG. 1B.

Medical user computational device 144 now also preferably comprises an encryption component 602, for encrypting information that is transmitted from medical user computational device 144 and decrypting information that is received by medical user computational device 144. Encryption component 602 also preferably supports encryption protocols that may be used for communication with a blockchain network 604. Server gateway 120 now also preferably comprises a blockchain node 604A, that is a node of blockchain network 604. Optionally, server gateway 120 only comprises an interface that communicates with a separate computational device handling communication with blockchain network 604 (not shown).

Blockchain network 604 preferably stores at least information about clinical trial protocols as described herein, including without limitation clinical trial frameworks that are suitable for different types of medical interventions; parameters for clinical trial measurements and outcomes; complete protocols and protocol “recipes” and the like. Optionally, each such building block and/or complete protocol features one or more comments or suggestions, for example regarding previous attempts to employ same in a clinical trial, and whether such attempts met with success, or had problems with patient compliance or other drawbacks.

These building blocks for clinical trial protocol and/or complete protocols are preferably stored on blockchain network 604 to permit greater access to clinicians and others involved in a clinical trial, as well as greater control to such access. In regard to access, clinicians and others may access blockchain network 604 from a variety of locations and through a variety of systems, optionally automatically, such that separate institutional and/or individual credentials may not be required. However, preferably blockchain network 604 supports control in terms of payment, such that a smart contract may be used to authorize access through a particular credential and/or computational device upon receipt of payment.

Optionally the building blocks for clinical trial protocol and/or complete protocols are not stored directly on blockchain network 604, but rather a pointer to an off-chain storage or storage system is stored on blockchain network 604. Through encryption and decryption for example, controlled access may still be maintained through blockchain network 604, in which upon payment for example, a decryption key may be provided to the purchaser, along with a pointer to the information purchased.

Blockchain network 604 may also optionally record meta data, such as how many people answered the questionnaire, how many people were signed up, translations etc.

FIG. 7 shows an exemplary, illustrative, non-limiting system incorporating blockchain according to at least some embodiments of the present invention. In a system 700, medical user computational device 144, user computational devices 102A-102C, and medical information computational device 140, each again communicates with server gateway 120 through computational network 116, as shown with regard to FIG. 4. Components having the same reference number have the same or at least similar function as for FIG. 4. Each such user computational device 102A-102C preferably is in communication with a wearable and/or comprises one or more sensors (not shown).

Various components of blockchain network 604 are shown as described in FIG. 6. Optionally an encryption component may be present at each device in system 700 (not shown). System 700 preferably additionally comprises a protocol computational device 702 and a vendor computational device 704, each of which preferably comprises or is in communication with a blockchain node 604A or 604B as shown.

Protocol computational device 702 may control access to clinical trial components and/or complete protocols, as described with regard to FIG. 6. For example, medical user computational device 144 may request access, optionally through a purchase, from protocol computational device 702. Such a purchase would then preferably be recorded on blockchain 604, including what was purchased and the identity of the purchaser, and optionally also any ties to particular clinical trials (if known). Optionally, medical information computational device 140 would request to record comments or add information to the protocol and/or its component through protocol computational device 702.

For example, and without limitation, a clinician or other personnel may be required to pay for access to a license to a questionnaire, such that protocol computational device 702 providing DRM (digital rights management) control through blockchain network 604. Optionally micropayments could be made through a smart contract on blockchain network 604, and/or through a pay per use system, rather than only accepting a single large payment for complete access. Optionally different rates could be charged, and different permissions could be provided for a commercial clinical trial as opposed to one being operated by a non-profit organization. The license terms and conditions may also be stored on and operated through blockchain network 604, for example for automatic permissions and payments.

Protocol computational device 702 may also provide version control for different versions of the clinical trial protocol and/or protocol parameters or building blocks. For example, optionally the most recent accepted version of a particular protocol and/or parameters or building blocks would be provided through blockchain network 604, so that clinicians always have access to the most recent information.

Vendor computational device 704 may accept payment and/or set prices for protocols, in place of or in addition to protocol computational device 702. Vendor computational device 704 may also control access to or provide equipment, such as wearables or software for example, for supporting the clinical trial execution and/or protocol development. Preferably, information about such access or equipment provision would be recorded on blockchain 604, including what was purchased and the identity of the purchaser, and optionally also any ties to particular clinical trials (if known). Comments to the vendor, and/or added information about the access and/or equipment provision, may also be recorded on blockchain 604.

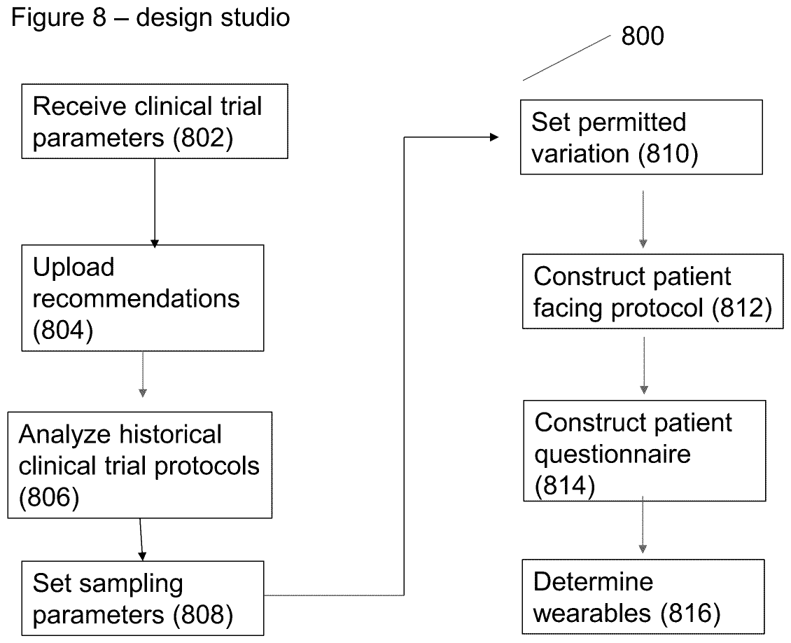

FIG. 8 shows an exemplary, non-limiting flow for a clinical trial protocol design studio. As shown in a flow 800, the process begins by receiving clinical trial parameters at 802. As described above, the basic required clinical trial parameters are known in the art, having been widely accepted by health authorities internationally. A non-limiting example of such a set of parameters are described in the ICH Guideline for Good Clinical Practice ICH E6 (R2) (https://ichgcp.net/6-clinical-trial-protocol-and-protocol-amendments). Such information includes but is not limited to the identity of the clinical trial sponsors, who are responsible for the conduct of the trial; the description of the product, device or service being tested; a description of the population under trial (women vs men, or a geriatric vs a pediatric population); references to background information; safety details; trial parameters and how they will be measured; and desired trial outcomes that would mark the trial as a success.

These parameters are optionally and preferably at least partially provided according to parameters of successful clinical trial protocols. For example, for trials involving wearables, preferably details are provided regarding how such wearables were tested and the parameters involved. As described herein, preferably an AI engine uploads such information, at least from prior trials but optionally also according to details provided by the trial designer or sponsor. For the latter, preferably at least information is provided regarding the service, therapy or device being tested, although optionally other such parameters are provided by the trial designer or sponsor.

Preferably such information includes reading and/or interpreting a clinical trial protocol, a technical documentation (User requirement Specification) or a structured input file (i.e. CDISC ODM) and extract the configuration parameters for the patient app or other software. Optionally pre-configured solutions based on previous projects and publicly available data about patient journeys and other ethnographical data may be used.

At 804, recommendations are also uploaded. These recommendations are applied according to the clinical trial parameters, such that the recommendations would differ according to the service, therapy or device being tested, and/or the population under test, and so forth. At 806, historical clinical trial protocols are analyzed. These are preferably complete protocols which include such items as to the degree of success of the trial and so forth. Having the complete protocols available provides the advantage of being able to analyze the protocol as a whole, to determine which strategies are successful.

At 808, sampling parameters are set. These sampling parameters relate to the frequency of obtaining data from the patient, whether with regard to diagnostic tests such as blood or urine tests, qualitative information such as questionnaires to be filled out, and/or data from wearables, smart phones or other sensors that are worn by or associated with the patient. For example, wearable devices generate data in three dimensions, for example with an accelerometer. Such wearables may generate up to 6060 data points per second, although many may generate less than that, such as for example 256 data points per second. Optionally, the data is obtained in snippets of specific intervals, such as six second intervals or multiple intervals within a longer period of time, such that not all data is obtained and/or analyzed. This type of analysis and data periods need to be set through the clinical trial protocol. Optionally, the data is plotted out and is then analyzed by the AI engine as an image, through computer vision protocols.

At 810, permitted variation is set. For example, the patient may be permitted to ingest a pill in the morning between 6 am and 9 am. Other treatments may have a longer period of time (at least once per day or week, for example) or a shorter period of time (hourly for example). However preferably a window of time is set, during which the treatment or other action is to be performed by the patient. Such permitted variation enables the clinical trial protocol to adapt the time windows and alarms for each patient (within permitted parameters) to make it easier for patients to comply with the protocol requirements. Such adaptation may be performed on the fly, through an analysis provided by the AI engine of the behaviors of the patient.

At 812, the patient facing protocol is constructed. Preferably the AI engine constructs the protocol from the provided information and also includes recommendations for the protocol. If the AI engine does not have sufficient information, then problematic areas in the protocol are flagged. The AI engine pulls together the potentially available data and the clinical trial parameters to construct the patient facing protocol, including for example the requirement for questionnaires and other types of data. Optionally a trained human being reviews the protocol before it is accepted.

At 814, the patient questionnaire is preferably constructed. Such questionnaires enable qualitative information to be obtained, as well as some types of quantitative information that may not be easily captured through other methods. Such questionnaires may be set up as diaries, preferably electronically. For example, the patient may include information such as when they took the medication or applied the product or service, and also optionally if and when they took any other medication including rescue medication. In cases where rescue medication or another type of rescue therapy is available, the questionnaire is preferably constructed to ask whether the patient used such a rescue medication or therapy. Preferably, alongside the questionnaire, the AI engine would generate an algorithm in the background to monitor or to calculate a compliance score.

In regard to the questionnaire, optionally the percent change of a parameter is considered, such as for example the level of pain, the frequency of rescue medication, how the patient feels overall and the patient's perceived activity levels. This information is preferably combined with the wearable data to show actual activity levels and how that corresponds to perceived activity levels. Such requests for information may be determined according to a type of protocol, such as a clinical trial protocol for patients suffering from rheumatoid arthritis as opposed to high cholesterol for example.

Optionally the questionnaire includes branching logic for a questionnaire, alarm sequences and time windows for answering. Preferably the AI engine also determines which questions are required, when a human or another system needs to be alerted and the flow for questions. Also optionally information is taken from a questionnaire library.

At 816, optionally one or more wearables are selected for the patient to use, in order to obtain actual activity data. Optionally such wearable data is reviewed by a doctor or other medical personnel on a periodic basis. Alternatively or additionally, the AI engine optionally reviews the wearable data continually or continuously, to determine one or more data points from the patient's activities and/or other physiological parameters. When one or more wearables are being selected, preferably the selection parameters include the role of wearable device data to drive certain behaviors, such as being too active or not active enough, for example post surgery. The wearable may also be selected to trigger a particular behavior, for example with an alarm, and then to ask one or more questions for context. Geolocation may be considered for context awareness, for example whether a patient should not be traveling or at a shop, as well as for example air quality.

Optionally the wearables may be selected and analyzed to guard against fraud, such as for example placing the wearable on another human or an animal. Fraud may also occur through enrollment of non-existing patients to a trial, which can also be determined according to wearable analysis. For example, physiological signals such as ECG and/or movement data may be analyzed for the data based “signature” of a patient. The wearables may also be selected and/or analyzed to avoid including errors, and/or to recover or add back missing data in the case of such errors.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for monitoring patient compliance during a clinical trial, comprising:
a user computational device accessed by a patient enrolled in a clinical trial;
one or more wearable devices in communication with said user computational device, said wearable devices comprising sensors for continuously monitoring patient behavior and physiological parameters during said clinical trial;
a server in communication with said user computational device through a computer network, said server comprising:
a database for storing clinical trial parameters including permitted time windows for patient actions; and
an AI engine configured to:
receive real-time data from said wearable devices indicating patient behavior and physiological parameters;
analyze said real-time data to determine patient compliance with clinical trial protocol requirements;
detect when patient behavior deviates from expected patterns based on said clinical trial parameters;
automatically adjust timing of alarms and prompts sent to said user computational device within said permitted time windows based on analysis of individual patient behavior patterns; and
generate protocol adjustments in response to detected compliance issues or routine disruptions while maintaining scientific rigor of said clinical trial;
wherein said system establishes a dynamic feedback loop between continuous patient monitoring through said wearable devices and adaptive protocol management, enabling real-time adjustment of patient-facing instructions based on actual patient behavior data collected between clinical visits.

2. The system of claim 1, wherein one or more wearable devices is selected from the group consisting of an accelerometer, a step counter, a GPS or equivalent sensor, actigraphy devices, insoles with one or more sensors, pulse oximeter, heart rate monitor, an airflow peak flow measuring device, brain wave monitor (EEG), an ECG (electrocardiogram).

3. The system of claim 1, wherein one or more wearable devices is selected from the group consisting of electronic weighing scales, blood glucose or other blood component measurement device, a needle tracking and/or disposal monitoring device, an ingestible sensor, and/or environmental monitors such as movement triggered cameras or sensors, air quality measurement devices, refrigerators that monitor food withdrawal or insertion, or video monitors.

4. The system of claim 1, further comprising a patient computational device for receiving input information from a patient or caregiver, wherein said input information comprises a patient questionnaire and wherein said patient questionnaire is constructed by the AI engine from said clinical trial protocol.

5. The system of claim 4, wherein said user computational device and said patient computational device are the same computational device.

6. The system of claim 1, wherein said user computational device further comprises a memory for storing a plurality of instructions and a processor for executing said instructions, wherein said processor executes said instructions to operate a user app interface, wherein patient-related information is entered through said user app interface; wherein said patient related information is selected from the group consisting of a subjective answer to a question, answers to a questionnaire, data from a wearable and a combination thereof; wherein said patient related information is transmitted to said server.

7. The system of claim 1, wherein said server comprises a processor and a memory with machine readable instructions, wherein execution of said instructions by said processor executes functions of said AI engine.

8. The system of claim 1, wherein the one or more wearables trigger a particular behavior with an alarm, for performing sampling from the patient according to at least one sampling parameter.

9. The system of claim 8, wherein the sampling parameters include the frequency of obtaining data from the patient.

10. The system of claim 9, wherein the data obtained from the patient includes diagnostic tests comprising at least one of blood or urine tests.

11. The system of claim 9, wherein the data obtained from the patient includes data from wearables, smart phones, or other sensors associated with the patient.

12. The system of claim 9, wherein the one or more wearable devices generate data in three dimensions using an accelerometer.

13. The system of claim 9, wherein the data is obtained intermittently.

14. The system of claim 9, further comprising plotting out the obtained data and analyzing the obtained data by said AI engine.

15. The system of claim 9, wherein data from the one or more is analyzed to guard against fraud.

16. The system of claim 15, wherein data from the one or more wearables is analyzed to detect enrollment of non-existing patients to a trial by analyzing physiological signals from a wearable or other sensor to specifically identify a patient.

17. The system of claim 9, wherein data from the one or more wearables is analyzed to avoid including errors and/or to recover or add back missing data in the case of such errors.

18. A method for monitoring patient compliance during a clinical trial through a user computational device accessed by a patient enrolled in a clinical trial; one or more wearable devices in communication with said user computational device, said wearable devices comprising sensors for continuously monitoring patient behavior and physiological parameters; and a server in communication with said user computational device through a computer network; the method comprising:
continuously collecting real-time data from said wearable devices indicating patient behavior and physiological parameters during said clinical trial;
transmitting said real-time data from said user computational device to said server through said computer network;
analyzing, by an AI engine on said server, said real-time data to determine patient compliance with clinical trial protocol requirements;
detecting, by said AI engine, when patient behavior deviates from expected patterns based on clinical trial parameters stored in a database;
automatically adjusting, by said AI engine, timing of alarms and prompts sent to said user computational device within permitted time windows based on analysis of individual patient behavior patterns;
generating, by said AI engine, protocol adjustments in response to detected compliance issues or routine disruptions while maintaining scientific rigor of said clinical trial; and
establishing a dynamic feedback loop between continuous patient monitoring through said wearable devices and adaptive protocol management, enabling real-time adjustment of patient-facing instructions based on actual patient behavior data collected between clinical visits.

19. A system for monitoring patient compliance during a clinical trial, comprising:
a user computational device accessed by a patient enrolled in a clinical trial;
one or more wearable devices in communication with said user computational device, said wearable devices comprising sensors for monitoring patient behavior and physiological parameters;
a server in communication with said user computational device through a computer network, said server comprising:
a database for storing clinical trial parameters; and
an AI engine configured to automatically construct a patient-facing protocol from said clinical trial parameters, said patient-facing protocol comprising specific instructions that determine when and how said patient is expected to interact with said wearable devices, when to complete questionnaires, and when to take other prescribed actions during said clinical trial;
wherein said AI engine is further configured to:
monitor real-time data from said wearable devices to determine patient compliance with said patient-facing protocol instructions;
detect when patient behavior deviates from expectations defined in said patient-facing protocol;
automatically adjust timing of alarms and prompts sent to said user computational device within permitted time windows specified in said patient-facing protocol based on analysis of individual patient behavior patterns; and
dynamically modify said patient-facing protocol instructions in response to detected compliance issues or routine disruptions while maintaining scientific rigor of said clinical trial;
wherein said patient-facing protocol serves as the operational framework that governs all patient interactions with said wearable devices, questionnaire completion schedules, and other trial-related actions, and wherein said system establishes a dynamic feedback loop between patient behavior monitoring and adaptive modification of said patient-facing protocol instructions.

20. The system of claim 19, wherein the server comprises a database for storing clinical trial parameters and an AI engine for analyzing the medical information, the clinical trial protocol and the clinical trial parameters, and for constructing the patient-facing protocol; wherein said AI engine outputs the patient-facing protocol for implementation; wherein the AI engine is configured to: analyze the medical information, the clinical trial protocol and the clinical trial parameters;
construct the patient-facing protocol by extracting key information from the medical information, the clinical trial protocol and the clinical trial parameters, using natural language processing, and constructing the patient-facing protocol from extracted key information based on previous similar clinical trials; and output the constructed patient-facing protocol for implementation;
wherein the patient-facing protocol comprises at least one instruction for an action to be taken by a patient enrolled in a clinical trial being operated according to the clinical trial protocol; wherein said at least one instruction comprises a permitted variation in regard to a time window for executing said action by said patient; wherein a plurality of time windows and alarms are adapted for each patient within permitted parameters.

21. The system of claim 20, wherein the AI engine selects and determines the role of the one or more wearables as part of the patient-facing protocol from said extracted key information; wherein said selected wearables are determined specifically by the AI engine according to the clinical trial protocol, but wherein the clinical trial protocol does not specify a particular wearable or other sensor.

22. The system of claim 21, wherein selection parameters for the one or more wearables include the role of wearable device data to drive at least one behavior of the patient.

23. A method for monitoring patient compliance during a clinical trial through a user computational device accessed by a patient enrolled in a clinical trial; one or more wearable devices in communication with said user computational device, said wearable devices comprising sensors for monitoring patient behavior and physiological parameters; and a server in communication with said user computational device through a computer network; the method comprising:
automatically constructing, by an AI engine on said server, a patient-facing protocol from clinical trial parameters stored in a database, said patient-facing protocol comprising specific instructions that determine when and how said patient is expected to interact with said wearable devices, when to complete questionnaires, and when to take other prescribed actions during said clinical trial;
implementing said patient-facing protocol as an operational framework that governs all patient interactions with said wearable devices, questionnaire completion schedules, and other trial-related actions;
monitoring, by said AI engine, real-time data from said wearable devices to determine patient compliance with said patient-facing protocol instructions;
detecting, by said AI engine, when patient behavior deviates from expectations defined in said patient-facing protocol;
automatically adjusting, by said AI engine, timing of alarms and prompts sent to said user computational device within permitted time windows specified in said patient-facing protocol based on analysis of individual patient behavior patterns;
dynamically modifying, by said AI engine, said patient-facing protocol instructions in response to detected compliance issues or routine disruptions while maintaining scientific rigor of said clinical trial; and
establishing a dynamic feedback loop between patient behavior monitoring and adaptive modification of said patient-facing protocol instructions.
